(19) United States
(12) Patent Application Publication
(10) Pub. No.: US 2026/0207392 A1
(43) Pub. Date: Jul. 23, 2026

(54) APPARATUS FOR DETERMINING A HYGIENE PROPERTY FOR A SANITARY PRODUCT

(71) Applicant: BASF SE, Ludwigshafen am Rhein (DE)

(72) Inventors: Thomas DANIEL, Ludwigshafen am Rhein (DE); Olaf HOELLER, Ludwigshafen am Rhein (DE)

(21) Appl. No.: 19/157,789

(22) PCT Filed: Mar. 8, 2024
(86) PCT No.: PCT/EP24/56166

§ 371 Date: Aug. 19, 2025

(30) Foreign Application Priority Data
Mar. 9, 2023 (EP) 23160847.2

(51) Int. Cl. A61F 13/15; G16C 60/00

(52) U.S. Cl. CPC A61F 13/15772; G16C 60/00; A61F 2013/15853

(57) ABSTRACT

Disclosed herein is a method for determining a hygiene property for a sanitary product. The sanitary product includes a layer of superabsorbent material. In the method, a) one or more absorption properties of a superabsorbent material, and b) a dosing model for the sanitary product including the dosing of a predetermined liquid with time at the super-absorbent material layer of the sanitary product are received. A sanitary product property determination model for determining a hygiene property of the sanitary product based on the absorption properties and on the dosing model is utilized. Control data for controlling a production process of the sanitary product and/or the superabsorbent material based on the determined hygiene property of the sanitary product is generated.

### FIELD OF THE INVENTION

The invention relates to an apparatus, a method and a computer program product for determining a hygiene property for a sanitary product. Moreover, the invention refers to a system, a method and a computer program product for controlling a production of a sanitary product and/or a superabsorbent material.

### BACKGROUND OF THE INVENTION

In many modern sanitary products, superabsorbent materials, often in form of superabsorbent particles, are utilized for fluid absorption. However, even for an expert it is often difficult to predict how a respective superabsorbent material impacts a performance parameter, in particular, a hygiene property, of a sanitary product. Thus, to present sanitary products are developed in a trial and error fashion and utilizing incremental development techniques. Thus, it would be advantageous if a hygiene property of a sanitary product could be predicted in a fast and accurate manner for controlling a production process of a sanitary product and/or a superabsorbent material utilized in the sanitary product.

### SUMMARY OF THE INVENTION

It is an object of the present invention to provide an apparatus, a method and a computer program product that allow for an improved determination of a hygiene property of a sanitary product that allows for an improved controlling of a production process of the sanitary product and/or the superabsorbent material, in particular to produce the sanitary product and/or the superabsorbent material continuously or batch-wise while meeting predetermined hygiene properties.

In a first aspect of the invention, an apparatus is presented for determining a hygiene property for a sanitary product, wherein the sanitary product comprises a layer of superabsorbent material provided in form of superabsorbent particles, wherein the apparatus comprises one or more processors configured to i) receive a) one or more absorption properties of a superabsorbent material, and b) a dosing model for the sanitary product comprising the dosing of a predetermined liquid with time at the superabsorbent material layer of the sanitary product, ii) utilizing a sanitary product property determination model for determining a hygiene property of the sanitary product based on the absorption properties and on the dosing model, and iii) generating control data for controlling a production process of the sanitary product and/or the superabsorbent material based on the determined hygiene property of the sanitary product.

It has been found by the inventors that it is possible to utilize a property determination model to predict a hygiene property of a sanitary product based on the absorption properties of a superabsorbent material for a predetermined liquid in the sanitary product and based on a dosing model indicating the dosing of the predetermined liquid with time at a superabsorbent material in a layer of the sanitary product. Since the model is only based on these two input parameters, the prediction of the hygiene property becomes efficient without having to first gather a huge amount of measurement data. This allows for a fast and computationally inexpensive prediction such that controlling data for controlling the production process of the sanitary product, for instance, for directly manipulating a production process of the sanitary product and/or a superabsorbent material, can be generated based on the respectively determined hygiene properties.

Generally, the apparatus can refer to any general or dedicated computing device adapted to perform the functions of the apparatus, for example, by executing a respective computer program. In particular, the apparatus can be realized in any form of soft- and/or hardware that causes the general or dedicated computing device to perform the functions as defined above. Moreover, the apparatus can be realized in form of a standalone device, for instance, in form of a dedicated hardware, or by being provided on a respective computer system of a user, but can also be realized in form of a network of computers or processors, for instance, in a shared computation regime like cloud computing, network computing, etc. in which more than one computer or processor can provide the functions of the apparatus.

Generally, the apparatus is adapted for determining a hygiene property for a sanitary product. The sanitary product can be any product that is used for private hygiene reasons, e.g. for excretions from humans or pets. In particular, the sanitary product is configured for absorbing bodily fluids. Preferably, the sanitary product refers to a diaper. However, the sanitary product can also refer to any other product configured for absorbing bodily fluids, like a female hygiene product, a medical hygiene product, etc. The hygiene property can refer to any property of the sanitary product that influences the hygiene of the sanitary product. In particular, since the sanitary product is a fluid absorption product, the hygiene property refers to a property indicative of the fluid absorption of the sanitary product. Preferably, the hygiene property is indicative of the amount of free liquid in the sanitary product. More Preferably, the hygiene property that is indicative of the amount of free liquid in the sanitary product refers to the maximum amount of free liquid in the sanitary product after a certain amount of applied fluid and/or the time of absorption, i.e. the time needed for absorbing a certain amount of free fluid until a predetermined minimum amount of a free liquid is reached. Preferably, the predetermined minimum amount of free liquid is zero so that no free liquid is present in the hygiene product at the end of the absorption process. However, in some applications a certain amount of free liquid even for extended periods of times can be acceptable, such that in these cases the respective minimum amount of free liquid can be above zero and set to the maximum amount of free liquid tolerable for extended time period in this application. Another preferred hygiene property is an acquisition time of the liquid in the hygiene product, wherein the acquisition time is the time until a liquid is completely collected in the hygiene product. In an embodiment, the hygiene property comprises or can be derived from a free liquid and/or an acquisition time.

In order to absorb a fluid, the sanitary product comprises a layer of superabsorbent material provided in form of superabsorbent particles. Superabsorbent materials are materials that can absorb and retain large amounts of aqueous liquid relative to their own mass. For example, for deionized and distilled water a superabsorbent material can absorb up to 1000 times its own weight. For practical applications in hygiene a superabsorbent material absorbs at least 15 g/g, typically at least 20 g/g, preferably at least 25 g/g and most preferably at least 30 g/g, but not more than 120 g/g, preferably not more than 100 g/g, more preferably not more than 80 g/g, most preferably not more than 60 g/g of a 0.9 wt. % saline solution (NaCl) in the absence of external pressure, e.g. in the tea bag method CRC. Absorption by a superabsorbent polymer is particular useful as compared to conventional absorbers like cellulose fluff because even under use pressure in the hygiene product the absorbed liquid will not be released, and the skin of the wearer will be kept dry. In most cases, the superabsorbent material comprises a superabsorbent polymer (SAP) often provided in form of a plurality of particles forming the superabsorbent material. A superabsorbent polymer is typically comprised of hydrophilic, and ionic group carrying polymer chains with high molecular weight and these polymer chains are interconnected to render the superabsorbent polymer water-insoluble. The ionic groups are typically-COOH, but can also be based on sulfur (—SO₃H) or phosporous as a provider of acidity. These groups are partially neutralized to achieve a skin-friendly pH on the wearer's skin, typically pH=4.0-7.5, preferably pH=5.0-6.5. As means of neutralization any alkali metal based neutralization agent (Li, Na. K, Rb, Cs) can be used but preferably Na is used in hygiene applications. Neutralization agents typically used are alkali metal salts of the hydroxides, carbonates, and hydrogencarbonates and mixtures thereof. Such neutralization agents can be used in form of a melt, a powder, a liquid, or as aqueous solutions, or as a mix of two or more of these physical forms in a sequential or concurrent neutralization process. Examples for such superabsorbent polymers are cross-linked poly-(meth)acrylic acid sodium salt, cross-linked poly-itaconic acid sodium salt, polyacrylamide copolymer, ethylene maleic anhydride copolymer, cross-linked carboxymethyl cellulose, cross-linked starch derivatives and carboxymethyl starch, polyvinylalcohol-grafted or starch-grafted cross-linked partially neutralized polyacrylic acid salts, polyvinyl alcohol copolymers, cross-linked polyethylene oxide, etc. Also, copolymers of acrylic acid, maleic acid, itaconic acid can be used and may be combined with copolymerized non-ionic hydrophilic or hydrophobic monomers. Preferable are partially neutralized cross-linked poly-acrylic acid and poly-itaconic acid salts as well as their copolymers. More preferable are partially neutralized cross-linked poly-acrylic acid and poly-itaconic acid salts as well as their copolymers of which the raw materials have been derived from biological sources, e.g. plants, microbes, algae, fungi. Most preferable are partially neutralized cross-linked poly-acrylic acid and poly-itaconic acid salts as well as their copolymers and superabsorbent production processes that produce the inventive superabsorbent polymer with a carbon footprint as low as possible.

The apparatus then comprises one or more processors that can perform the functions of the method described in the following. In a step, for instance, performed by a receiving unit of the apparatus realized by one or more processors, one or more absorption properties of the superabsorbent material that is to be utilized in the sanitary product are received. In particular, the absorption properties of a superabsorbent material can be received, for instance, by accessing a storage unit on which respective absorption properties are already stored. Such absorption properties of a superabsorbent material can be stored, for instance, on a database or in a superabsorbent material library which is filled with a plurality of different superabsorbent materials and associated absorption properties. Such a database or library can be generated by performing respective experiments and measurements of absorption properties of a plurality of superabsorbent materials or by respective simulations of absorption properties of the superabsorbent material. The receiving of the one or more absorption properties of the superabsorbent material can then, for instance, comprise receiving a superabsorbent material, for instance, via a user input, or via an automatic determination, and accessing the respective database or library to receive the respective absorption properties stored on the database or library in association with the received superabsorbent material. However, the one or more absorption properties can also be received, for instance, via an input unit, from a user providing the respective absorption properties. Moreover, the respective absorption properties of a superabsorbent material can also be directly received from a respective measurement setup in which the respective absorption properties have been measured for the superabsorbent material.

Preferably, the absorption property refers to at least one of an absorption capacity, a swelling kinetics and a permeability. An absorption capacity can be determined as any of a centrifuge retention capacity (CRC), a free swelling capacity (FSC), and an absorption capacity against pressure (AAP). Generally, CRC and FSC are determined without external pressure, i.e. with 0.0 psi. in both cases the higher the respective CRC or FSC value the more fluid can be absorbed by a particle. A swelling kinetics can refer to any of VAUL, T20 and Vortex. For irregular shaped, rough surface particles these three quantities are correlated. For particles with a regular, smooth surface or round shape the Vortex may not be correlated with the other quantities. Generally, the faster the swelling of the particle the smaller is the respective value of any of the quantities. The permeability (SFC) is correlated nonlinearly with the CRC-capacity. Generally, the higher the permeability value the better the fluid is distributed in a particle convolute. The CRC can be interpreted as the mere absorption capacity of the superabsorbent material, the SFC can be interpreted as the mere permeability of the open pores in the swollen gel bed of the swollen superabsorbent material, the FSC, or AAP can be interpreted as absorbent capacities of the superabsorbent material under defined external pressure conditions and do each comprise a contribution from the swollen superabsorbent material and a contribution from the partially or completely liquid filled pores in the resulting gel-bed (interstitial liquid), and the T20, Vortex, and VAUL characteristic swelling time can be interpreted as kinetic swelling speed parameters describing the rate of swelling and may include additional effects other than absorption rate—for example particle morphology and surface stickiness can affect these measurements. The rate at which liquid is absorbed into the swelling superabsorbent material particle is defined as absorption rate. These properties and respective methods for determining the properties are further described below. Various other forms of properties or methods for determining respective properties exist and can also be used as technical application property according to the present invention. Other methods may differ, for example, in equipment dimensions, e.g. smaller or larger AAP-cells or permeability-cells, in the handling techniques, in a test liquid, e.g. water, artificial urine instead of saline, in the swelling times, e.g. 1, 3, 5, or 10 min instead of 30 min. Generally, the selection of the absorption properties can depend on the respective intended application of the hygiene product, for instance as diaper or medical product. A particular useful method for measuring an absorption property is described in WO 2021/001221 which allows to determine time dependent swelling profiles of superabsorbent polymers at varied external pressures. In this way a characteristic fingerprint-curve is obtained that can be used to define an absorption property. Particularly useful is an inline analytics method as disclosed in WO 2020/109601 which enables to determine absorption properties by Raman-spectra analytics inside the production process. Combination of such an inline technique with an inline particle size determination enable highly efficient use cases with the present invention. In a particularly preferred embodiment, the one or more absorption properties of the superabsorbent material are indicative of at least one of a permeability, an absorption capacity and an absorption speed. Even more preferably, the absorption property is a permeability of the superabsorbent material. Most preferably the absorption property is a combination of a permeability and an absorption speed.

The superabsorbent material is provided in form of superabsorbent particles comprising a superabsorbent polymer. The size of the superabsorbent particles for most applications lies preferably between 100 μm and 850 μm. However, for some applications also larger or smaller superabsorbent particles can be suitable. Lately, narrower particle size distributions have been required for some hygiene products which are much more difficult and costly to produce and challenging to optimize their technical properties, for example with a lower particle size of 100, 150, 200, or 250 μm and an upper particle size of 700 μm, 600 μm, or 500 μm.

Preferably, each superabsorbent particle comprises a) an interconnected core and b) a surface cross-linked shell with a higher connectivity than the core. In this context, the terms “interconnected”, “connectivity” mean that the polymer chains of the core- or shell-part of the superabsorbent polymer particle are physically entangled, ionically or covalently crosslinked so that the respective parts of the superabsorbent particles are water-swellable but not water-soluble. A combination of such methods to interconnect the polymer chains is possible and typically found in superabsorbent particles. Ionic cross-linking can be achieved by multivalent cations, practical examples are Mg²⁺, Ca²⁺, Sr²⁺ Al³⁺, Ti⁴⁺, Zr⁴⁺. Covalent cross-linking can be achieved by addition of polymerizable di- or polyfunctional ethylenically unsaturated cross-linkers to the monomer mixture. Alternatively, this functionality can also be provided by groups that can undergo esterification or transesterification. Mixed functionalities in one molecule are also possible. For surface-cross-linking the same compounds as described above are useable. Typically, ionic cross-linkers and covalent cross-linkers are used in combination. Examples for core- and surface-cross-linking are described in WO 2019/197194 which is incorporated in here by reference. In the present invention it is understood that the shell and the core of the superabsorbent particles are linked to each other by physical entanglement or ionic or preferably by covalent crosslinking. Such a core-shell structure may break up the surface-shell of the superabsorbent particle when swelling but due to the connectedness of the shell to the core it will continue to exert physical forces onto the swelling core even if the shell is completely broken. Preferably, the superabsorbent particles refer to a post cross-linked superabsorbent polymer particle. Such a post cross-linked superabsorbent polymer particle comprises a crosslinked and thus interconnected core and is then provided in a post cross-linking process with a shell comprising a higher connectivity than the core while this shell is covalently bound to its underlying core. Optionally, a such provided superabsorbent particle can also be provided with an additional non-superabsorbent coating. Non-limiting examples of such coatings are polymers or polymer films to improve flowability or damage stability, powder coatings to prevent caking (silica, alumina, clay or other inorganic powders in their dry or hydrated forms), additives to prevent ageing or discoloration, additives that combat malodour, or functional coatings that react with the surface like Ca²⁺-, Mg²⁺, Al³⁺- and Zr⁴⁺-salts or their soluble hydroxides as for example published in WO 2019/197194.

Further, a dosing model for the sanitary product is received. Also in this case, the dosing model can be received by accessing a storage unit on which one or more dosing models are already stored, or by receiving an input, for instance, from a user via a user input. The dosing model comprises at least information on the dosing of a predetermined liquid with time at the superabsorbent material layer of the sanitary product. Thus, the dosing model provides information on the amount of fluid that reaches a superabsorbent material layer of the sanitary product with time in order to allow to simulate different application scenarios. For example, the dosing model can comprise one or more amounts of fluid at the superabsorbent material layer versus time curves that correspond to different application scenarios. Moreover, the dosing model, in particular the dosing of a predetermined liquid with time at the superabsorbent material layer, can also implicitly comprise information on the respective composition and structure of the sanitary product surrounding the layer of superabsorbent material. For example, specific layers utilized in sanitary products can delay fluid introduced into the sanitary product from reaching the superabsorbent material layer, whereas other compositions or structures of the sanitary product can lead to a fast concentration of fluid introduced into the sanitary product into the superabsorbent material layer. These compositions and structures of the sanitary product thus can influence the dosing of a liquid with time at the superabsorbent material layer. Depending on the envisioned application of the sanitary product, such respective influences can be taken into account and the dosing of the predetermined liquid with time at the superabsorbent material layer can be provided accordingly. For example, for different compositions and structures of a sanitary product measurements for respective application cases in a laboratory can be performed to determine the dosing of the predetermined amount and composition of liquid with time at the superabsorbent material layer. The respective information, i.e. the dosing model, can then be received, for instance, by indicating a respective sanitary product and a respective application case, for instance, via a user input, and then accessing a respective database on which corresponding dosing models for one or more application cases are stored. However, if the composition and structure has no strong influence on the dosing model, also a standard dosing model for one or more application cases can be provided and accessed for receiving the dosing model.

Moreover, one or more of the absorption properties can have an influence on the dosing model. In particular, a permeability of the superabsorbent material can determine the amount of liquid reaching the superabsorbent layer with time. Thus in a preferred embodiment the dosing model depends on the permeability of the superabsorbent material. In this case dosing models for different permeabilities can be stored on a respective storage and the receiving can comprise selecting based on the permeability of the superabsorbent material the respective dosing model from the storage. However, the receiving of the dosing model can also comprise modifying a standard dosing model based on a received permeability, for instance, by amending, if necessary, the amount of liquid reaching the superabsorbent layer with time based on the permeability of the superabsorbent material. Preferably, the dosing model does comprise the permeability of the superabsorbent material and the permeability provided by the sanitary product, for instance, if it comprises liquid managing elements capable to substitute for some or all of the superabsorbent material's permeability. Liquid managing elements can be embedded into the superabsorbent material layer or positioned above or underneath the superabsorbent material layer. Examples for embedded liquid managing elements are synthetic resin fibers, cellulose fibers, crosslinked cellulose fibers, porous nowovens, channel structures, profiled superabsorbent material distribution inside the core. Examples for adjacent liquid managing elements, usually placed on top, e.g. facing the wearers body, of the superabsorbent material layer, are acquisition distribution layers in form of apertured films, non-wovens, cross-linked cellulose fibers, polymer resin top-sheets and the like. Such liquid managing elements provide porosity to the superabsorbent material layer comprising the superabsorbent material or provide porosity adjacent to the superabsorbent material layer. This enables quick liquid distribution of the liquid across or inside the superabsorbent material layer. As a result, the superabsorbent material may be provided in terms of required permeability in exchange for more absorption capacity. Liquid managing elements can be used in combination with a permeability of the superabsorbent material. One or more liquid managing elements may be used in a sanitary product, in particular, a diaper. Preferably the respective dosing model takes such product features into account as described above.

Further, the apparatus then utilizes a sanitary product property determination model for determining a hygiene property of the sanitary product based on the absorption properties and on the dosing model. The sanitary product property determination model can be any model that allows for determining a hygiene property of the sanitary product based on the absorption properties and/or the dosing model. In particular, the sanitary product property determination model is a mathematical model. In an embodiment, the sanitary product property determination model can be a data driven model that has been parameterized, for example, based on historical measurement data of a hygiene property depending on the absorption properties and the dosing model. In particular, the term “data driven” defines that the model is mainly based on respective data input and not, for instance, on intuition, personal experience or knowledge. The property determination model can, in particular, be realized as any machine learning based model that is based on known machine learning algorithms, like neural networks, regression models, classification algorithms, etc.

Also, a white-box model evaluated with a non-linear regression optimizer can be used in the present invention. Such a machine learning model can be used as part of a comprehensive process control model, optionally, taking into account other formulation and process parameters, using other machine learning and artificial intelligence algorithms. Generally, such a machine learning based property determination model comprises one or more model parameters that can be determined based on respective training data. The parameterization of the property determination model is thus a determination of the values of the respective one or more model parameters based on the training data in a model training process. In particular, the determination model is parameterized such that it can determine the hygiene property of the hygiene product based on the dosing model and the absorption properties. Generally, for parameterizing the property determination model accordingly, respectively known training methods for parameterizing a given model can be utilized. In particular, optimization methods can be utilized to find an optimal fit of model parameters to the respective training data. For the training of the property determination model preferably historical data can be utilized as training data, for example, historical data comprising measurement data from respective measurements of hygiene properties of a plurality of hygiene products with respective different superabsorbent material layers subjected to different dosing scenarios.

However, in a preferred embodiment the sanitary product property determination model is based on ordinary or partial differential equations determining the influence of the dosing model and the absorption properties of the superabsorbent material on the hygiene property. Preferably, The apparatus according to any of the preceding claims, wherein the sanitary product property determination model comprises solving the following differential equation

$$\frac{dL}{dt} = - \frac{dQ}{dt} + \frac{dV}{dt} ,$$

wherein

$$\frac{dL}{dt}$$

describes the free liquid in the sanitary product with time,

$$\frac{dQ}{dt}$$

describes the absorption of fluid by the superabsorbent material with time depending on the absorption properties, and

$$\frac{dV}{dt}$$

describes the dosing of the fluid with time into the sanitary product.

Preferably, the sanitary product provides sufficient void space to acquire and distribute the penetrating liquid for situations when this penetration is faster than the ensuing liquid absorption by the superabsorbent polymer. It has been found by the inventors that in particular the above differential equation accurately describes as hygiene property a free liquid in the sanitary product with time and at the same time can be easily solved utilizing known and routine methods for solving differential equations with numerical algorithms. Non-limiting examples of useful algorithms are Euler and Runge-Kutta methods. In particular, compared with utilizing machine learning methods solving a respective differential equation directly is less computationally expensive and faster to implement.

Further, the apparatus is configured for generating control data for controlling a production process of the sanitary product and/or the superabsorbent material based on the determined hygiene property of the sanitary product. The control data can refer to any data that can in some way influence the production process of the sanitary product. For example, the control data can directly comprise respective information on production process parameters of the sanitary product and/or superabsorbent material. However, the control data can also comprise more general information that only in combination with, for instance, a production process control application allows for an amendment of production process parameters. Accordingly, the control data can be provided in any suitable format. For example, the control data can be provided in a format that allows a direct implementation of the control data in a production process controlling application. However, the control data can also be provided in a format that first has to be translated into a respective format. Generally, the control data can be provided directly for the control of the production process or can first be provided, for instance, to a user for a security check, wherein the control data is only implemented if the user accepts the control data. Preferably the method comprises relaying the control data to a production control system of the production process.

In an embodiment, the one or more received absorption properties are measured during a production process of the superabsorbent material and/or of the sanitary product compressing the superabsorbent material and wherein the generated control data is configured to control and/or monitor the production process based on the hygiene property determined from the one or more measured absorption properties. In particular, the determined hygiene property can be evaluated against a predetermined target hygiene property and the control data can be generated based on the comparison. For example, if the determined hygiene property deviates outside of pre-set boundaries from the target hygiene property a control and/or monitoring action can be performed, like notifying an operator, changing one or more production parameters, changing a formulation, changing the throughput in the polymerization reactor, modifying the gel comminution or the gel drying, stopping a chemical reaction or changing a milling or a sieving procedure, modifying the post cross-linking step or a coating step. The measured absorption properties can comprise a particle size distribution and/or crosslinking properties of the particles. Preferably, Raman spectroscopy is utilized for monitoring the chemical reaction of the crosslinking process of the superabsorbent particles during the production process and to determine a thickness of respective crosslinking shells of the superabsorbent properties. Optical measurements can be utilized for determining a respective particle size distribution during the production process. The one or more absorption properties can be measured continuously during the chemical production process for producing the superabsorbent material, wherein continuously refers to measuring in predetermined time intervals that are much smaller than the production process. Directly measuring the absorption properties of the superabsorbent particles during the production process allows to monitor the resulting hygiene property of the final product and to change process parameters if the hygiene properties deviate from a predetermined target. In particular, the in-situ measuring during the production process allows to avoid delays in the control of the process that would result from laboratory measurements of the absorption properties and thus leads to a direct control and monitoring. In an embodiment, the one or more processors are further configured for receiving a target hygiene property of the sanitary product and to compare the determined hygiene property with the target hygiene property and to determine i) the superabsorbent material as the target superabsorbent material for the sanitary product if the determined hygiene property lies within a predetermined range around the target hygiene property, and ii) a new superabsorbent material and repeating the determination of the hygiene property of the sanitary product using the new superabsorbent material if the determined hygiene property lies outside a predetermined range around the target hygiene property, and wherein the controlling of a production process is based on the determined target superabsorbent material. Preferably, determining of a new superabsorbent material comprises amending a size distribution of the particles forming the superabsorbent material and/or the amount of superabsorbent material in the layer. Generally, since the size distribution of the particles forming the superabsorbent material can have a huge influence on the absorption properties of the superabsorbent material amending the size distribution also allows to change the absorption properties of the superabsorbent material and thus potentially allows to meet the target hygiene properties. However, it is also possible to provide as new superabsorbent material a completely different superabsorbent material, for instance, a superabsorbent material with different composition or formed from a different superabsorbent polymer. Moreover, the new superabsorbent material can also be determined by determining a different amount of the superabsorbent material in the respective superabsorbent layer. Generally, since at least some absorption properties of a superabsorbent material depend on the amount of the superabsorbent material present in the layer, changing the amount of superabsorbent material can also change in particular these superabsorbent properties and thus can potentially allow to meet the respective target hygiene property. Moreover, all of the above described parameters can also be changed in combination. Generally, the determination of the new superabsorbent material can be based on respective predetermined rules and additionally or alternatively based on iterative methods for amending the respective property until the target hygiene property is met in predetermined limits. For example, predetermined rules can determine that during a first iteration the size distribution of the particles forming the superabsorbent material is amended, for instance, utilizing a respective gradient method for determining a new particle distribution in each iterative step, wherein if after a plurality of predetermined steps or if after reaching a predetermined particle size distribution limit, for instance, an upper or lower limit of the particle size, the predetermined rules can determine that for a next iteration the amount of superabsorbent material in the superabsorbent layer is amended in predetermined increments with each iterative step. If still after having reached a predetermined limit for the amount of superabsorbent material in the layer, for instance, a predetermined upper limit for the amount of superabsorbent material in the layer, the target is not met, the rules can further define that for the next iteration the superabsorbent material itself is changed to a different composition, wherein the iteration is then again performed with respect to the particle size distribution of the superabsorbent material.

In a preferred embodiment, further an indication on an amount of superabsorbent material in the sanitary product layer is received and the one or more absorption properties of the superabsorbent material are adapted based on the amount of superabsorbent material in the sanitary product.

In a preferred embodiment, the apparatus further comprises one or more processors that are configured for determining the one or more absorption properties of the superabsorbent material based on a received particle size distribution of the superabsorbent material utilizing an absorption property determination model, wherein the property determination model is a data-driven model that has been parameterized such that it is adapted to determine a technical application property of a superabsorbent particle based on the size of the particle, wherein the determined one or more absorption properties are then utilized in the hygiene property determination model. Preferably, the superabsorbent particles of the superabsorbent material comprise a superabsorbent polymer provided in form of i) an interconnected core and ii) a surface cross-linked shell with a higher connectivity than the core, and the provided absorption property determination model is further parameterized based on a core size and a shell size of the superabsorbent particles.

The one or more processors are then further configured to utilize an absorption property determination model for determining the absorption property of the superabsorbent material based on the particle size. In particular, the absorption property determination model is realized as a data-driven model that has been parameterized, for example, based on historical measurement data, such that it is adapted to determine an absorption property of a superabsorbent particle based on the size of the particle. Preferably, the absorption property determination model can be realized as any machine learning based model that is based on known machine learning algorithms, like neural networks, regression models, classification algorithms, etc. A white-box model evaluated with a non-linear regression optimizer is particularly useful in the present invention. Generally, the absorption property determination model comprises one or more model parameters that can be determined based on respective training data. The parameterization of the absorption property determination model is thus a determination of the values of the respective one or more model parameters based on the training data in a model training process. In particular, the absorption property determination model is parameterized such that it can determine an absorption property of a superabsorbent particle based on the size of the particle. Generally, for parameterizing the absorption property determination model accordingly, respectively known training methods for parameterizing a given model can be utilized. In particular, optimization methods can be utilized to find an optimal fit of model parameters to the respective training data. For the training of the absorption property determination model preferably historical data can be utilized as training data, for example, historical data comprising measurement data from respective measurements of absorption properties of superabsorbent particles or data derived from known physical relations of the absorption property and respectively measurable characteristics of the superabsorbent particle. In particular, the historical data comprises for a plurality of different particle sizes of a superabsorbent particle corresponding to one or more absorption properties.

Since the production process of a superabsorbent particle can have a high influence on the specific composition of the superabsorbent particle, for example, on the specific interconnectivity of the core and the shell of the superabsorbent particle, it is preferred that a respective historical data set for a specific production process for respectively produced superabsorbent material is utilized for parameterizing the absorption property determination model. This allows the absorption property determination model to provide a very accurate determination of the absorption property of the superabsorbent material produced in the specific production process. However, in other embodiments, the absorption property determination model can also be trained with a more general historical data set comprising data for superabsorbent materials utilizing different production processes, wherein the absorption property determination model can in such a case then learn to differentiate between superabsorbent materials produced in different production processes and the respective production process can be provided as further input to the absorption property determination model. Moreover, the absorption property determination model can be trained such that it can determine one absorption property of a superabsorbent material, but can also be trained to predict more than one absorption property of a superabsorbent material.

In a preferred embodiment, the utilized absorption property determination model is further parameterized based on a core size and a shell size of the superabsorbent particle. Surprisingly, it has been found by the inventors that further parameterizing the absorption property determination model based on a core size and a shell size of the superabsorbent particle allows for a particularly accurate determination of the absorption property. Moreover, parameterizing the absorption property determination model further based on a core size and a shell size of the superabsorbent particle allows to separate the respective influence of each of these parameters on the absorption property. Generally, the core size and the shell size of a superabsorbent particle depend on the particle size, i.e. the penetration depth of substances used for the post cross-linking process is substantially the same for all particle sizes such that the size, i.e. volume, of the shell and the core depend mainly on the size of a particle. However, the general penetration depth of the post cross-linking substances and thus the thickness of the shell relative to the core depend on the utilized crosslinking procedure, for example, the utilized substances, pressure conditions, utilized additives, temperatures, etc. Thus, further separating the influence of the core and shell size on the absorption property allows not only for utilizing the size of the superabsorbent particles for controlling the absorption properties, but also allows to determine the influence of the shell size and core size on the absorption property and thus allows to also optimize the post cross-linking procedure for the superabsorbent particles with respect to the technical application property.

Preferably, the parameterizing of the utilized absorption property determination model comprises determining performance parameters quantifying a contribution of the core size and the shell size, respectively, of a superabsorbent particle to the absorption property. Utilizing a respective training data set that comprises for a plurality of superabsorbent particle sizes corresponding core sizes, shell sizes and absorption properties, allows to parameterize an absorption property determination model such that the influence of the core size and the shell size on the absorption property can accurately be quantified by determining the performance parameters. Preferably, the utilized absorption property determination model is based on the following relation between the absorption property and a size of a superabsorbent particle

$${var}_{Particle} = \frac{{V}_{shell} {var}_{shell} + {V}_{core} {var}_{core}}{{V}_{shell} + {V}_{core}}$$

wherein vₛₕₑₗₗ is a volume of the shell, and v_{core} is a volume of the core of the superabsorbent particle, wherein the volume of the shell and the volume of the core of the superabsorbent particle depend on the size of the superabsorbent particle, and wherein var shell and var core are the performance parameters quantifying the contribution of the core size and the shell size, respectively, and are determined during the parameterization of the property determination model, and wherein var Particle is indicative of the measured absorption property.

In an embodiment, the received particle size is a particle size distribution of the superabsorbent particles of the superabsorbent material. In this case, based on the particle size distribution, one or more particle size classes from predetermined particle size classes can be determined for which particles with respective sizes are present in the superabsorbent material. An absorption property can then be determined for the determined particle size classes and an overall application property of the superabsorbent material can be determined based on the determined absorption properties for the respective determined particle size classes and based on the particle size distribution. Generally, the particle size distribution of a superabsorbent particle can be received in form of any data information that is indicative of the particle sizes that are present in a statistically relevant sample of the superabsorbent material. For example, the particle size distribution can be provided in the form of a list of all sampled superabsorbent particles and corresponding sizes of the superabsorbent particles. However, the particle size distribution can also directly be provided in the form of a class distribution indicating for a plurality of particle size classes the amount of particles present in the respective class in a statistically relevant sample. Generally, a particle size class refers to a particle size range and is defined by a smallest particle size and a largest particle size defining the particle size range. The respectively predetermined particle size classes can then refer to the already utilized particle size classes if the particle size distribution is provided in the form of a class distribution, wherein in this case the determination of whether particles are present in a predetermined particle size class amounts to determining whether an amount of particles greater than zero is indicated by the particle size class distribution in a respective particle size class. However, the predetermined particle size classes can also be independent of any previously utilized particle size classes for providing a particle size class distribution. In this case, respective statistical methods can be utilized to determine for which of the predetermined particle size classes particles are present in the superabsorbent material. Moreover, if a list of particle sizes is provided as particle size distribution, the predetermined particle size classes can be utilized to sort the particle sizes accordingly and to determine for which predetermined particle size class at least one particle is present. Preferably, the particle size distribution, for instance, of waterabsorbent polymer particles, can be determined by the EDANA recommended test method No. WSP 220.3 (11) “Particle Size Distribution”. Also, optical methods, for example, laser diffraction, photographic analysis, an array of light-beams and spatial filter velocimetry (Parsum® probe) etc., can be favourably utilized, preferably, calibrated against the EDANA or corresponding ISO-test method based on a screening analysis. Such calibration may depend also on other particle properties except the particle size and is therefore carried out specifically for each product grade. In the present invention such calibrated methods are particularly useful as they can be used inline of the production process at one or more locations and provide the necessary particle size information in real time.

The absorption property for the determined particle size classes is then determined by utilizing the absorption property determination model for a respective particle size falling within the predetermined particle size classes. Generally, the absorption property can be determined by providing at least one particle size falling in a predetermined particle size class to the absorption property determination model and utilizing the determined absorption property as technical application property for all sizes falling within the determined particle size class. However, also the smallest and the largest size of particles falling within the respectively determined particle size class can be utilized as input to the absorption property determination model and the respectively determined absorption properties can be statistically combined, for instance, by averaging to determine an absorption property representing the respective determined particle size class. However, also other statistical methods can be utilized accordingly. The overall absorption property can then be determined based on the determined absorption properties for the respective determined particle size classes and based on the particle distribution. In particular, the amount of particles falling within a respective determined particle size class is taken into account in determining the overall absorption property. For example, a weighted averaging can be utilized for determining the overall absorption property based on the determined absorption properties, wherein the weights of the weighted averaging are determined based on the amount of particles of the particle distribution falling within a respective particle size class. For example, if more particles fall within a particle size class, the respective absorption property can be weighted higher than an absorption property corresponding to a particle size class with less particles. Moreover, also other known or learned relations can be taken into account, for instance, in the weights for determining the overall absorption property. For example, it can be determined that bigger particles have generally a higher influence on an overall absorption property than smaller particles. In this case, the weights for particle size classes with bigger particles can be provided higher with respect to the weights of particle size classes with lower size particles. However, for some absorption properties it can be determined that smaller particles have generally a higher influence than bigger particles. In this case, the weights for particle size classes with smaller particles can be provided higher with respect to the weights of particle size classes with bigger size particles. Typically, for absorption capacities with and without external pressure an arithmetic average can be utilized to predict the overall product properties from the individual particle size classes. For performance critical properties like swelling kinetics and liquid permeability this is not the case and rather complex mixing properties are found for blends of superabsorbent particles with different properties—for example as described in WO 2019/137833. This is due to the fact that for such blends not only the size-class-specific properties are relevant but also complex mixing phenomena based on the amount of particles present in the different size-classes show significant impact on the overall performance. While more finer particles can be beneficial to achieve fast liquid absorption, they can act antagonistic in terms of liquid distribution as they can quickly block fluid conducting pores. To predict such properties a weighted average value of the size-classes may be used that does not treat all particle sizes equally and can depend on the number of particles in each class. In the present invention, the weights are preferably experimentally determined by first measuring the superabsorbent polymer's overall performance properties and then after classifying into the respective discrete size classes by determination of the properties for each class. Mixing the respective classes in varied amounts with each other will allow conclusions towards the required weights. For such mixing one can use a design of experiments. The determined overall absorption properties are then utilized as absorption property for the respective particle size distribution in all further processing, in particular, for determining the hygiene property.

In a further aspect of the invention, a system is presented for controlling a production of a sanitary product and/or a superabsorbent material, wherein the sanitary product comprises a layer of the superabsorbent material provided in form of superabsorbent particles, wherein the system comprises i) an interface unit configured for receiving a) one or more production parameters utilized for producing the sanitary product and b) a target hygiene property of the sanitary product, and for determining one or more absorption properties of the superabsorbent material and/or a dosing model for the sanitary product based on the received one or more production parameters, ii) an apparatus according to any of the preceding claims, wherein the apparatus is configured to receive the absorption properties of the superabsorbent material and/or a dosing model from the interface unit and to determine the hygiene property, and further to compare the determined hygiene property with the target property and if the determined hygiene property lies outside a predetermined range around the target hygiene property generate control signals for amending one or more of the one or more production parameters utilized for producing the sanitary product and/or the superabsorbent material. Generally, the amendment of the one or more production parameters utilized for producing the sanitary product and/or the superabsorbent material can be generated according to predetermined rules based on process knowledge. For example, if a predetermined hygiene property lies above a predetermined range around a target hygiene property, the rules can indicate, for example, that specific production parameters should be decreased in predetermined increments until the hygiene property lies again within the predetermined range around the target hygiene property. Such rules can generally be based on experiments or previous experience with the production process.

In another aspect of the invention, a computer implemented method is presented for determining a hygiene property for a sanitary product, wherein the sanitary product comprises a layer of superabsorbent material provided in form of superabsorbent particles, wherein the method comprises the steps of i) receive a) one or more absorption properties of a superabsorbent material, and b) a dosing model for the sanitary product comprising the dosing of a predetermined liquid with time at the superabsorbent material layer of the sanitary product, ii) utilizing a sanitary product property determination model for determining a hygiene property of the sanitary product based on the absorption properties and on the dosing model, and iii) generating control data for controlling the production process of the sanitary product and/or the superabsorbent material based on the determined hygiene property of the sanitary product.

In another aspect of the invention, a computer implemented method is presented for controlling a production of a sanitary product and/or a superabsorbent material, wherein the sanitary product comprises a layer of the superabsorbent material provided in form of superabsorbent particles, wherein the method comprises i) receiving a) one or more production parameters utilized for producing the sanitary product and b) a target hygiene property of the sanitary product, ii) determining one or more absorption properties of the superabsorbent material and/or a dosing model for the sanitary product based on the received one or more production parameters, iii) performing a method according to claim 11 based on the received absorption properties of the superabsorbent material and/or a dosing model to determine the hygiene property, and, iv) compare the determined hygiene property with the target property and if the determined hygiene property lies outside a predetermined range around the target hygiene property generate control signals for amending one or more of the one or more production parameters utilized for producing the sanitary product and/or the superabsorbent material.

In another aspect of the invention, a computer program product is presented for determining a hygiene property for a sanitary product, wherein the computer program product comprises program code means for causing the apparatus as described above to execute the method as described above.

In another aspect of the invention, a computer program product for controlling a production of a sanitary product and/or a superabsorbent material, wherein the computer program product comprises program code means for causing the apparatus as described above to execute the method as described above.

It shall be understood that the method as described above, the apparatuses as described above, and the computer program products as described above have similar and/or identical preferred embodiments, in particular, as defined in the dependent claims.

It shall be understood that a preferred embodiment of the present invention can also be any combination of the dependent claims or above embodiments with the respective independent claim.

These and other aspects of the present invention will be apparent from and elucidated with reference to the embodiments described hereinafter.

### BRIEF DESCRIPTION OF THE DRAWINGS

In the following drawings:

FIG. 1 shows schematically and exemplary a system for controlling a production of a sanitary product and/or a superabsorbent material used in a sanitary product,

FIG. 2 shows schematically and exemplary a flow chart of a method for controlling a production of a sanitary product and/or a superabsorbent material utilized in the sanitary product,

FIG. 3a, b, c show schematically and exemplary illustrations of a one-dimensional, two-dimensional and three-dimensional model for determining a hygiene property of a sanitary product, respectively,

FIGS. 4a to 4d shows schematically and exemplary an application of the invention to the controlling processes for producing a sanitary product and/or a superabsorbent material, and

FIG. 5 shows schematically and exemplarily a utilization of Torricelli's law for a dosing model.

### DETAILED DESCRIPTION OF EMBODIMENTS

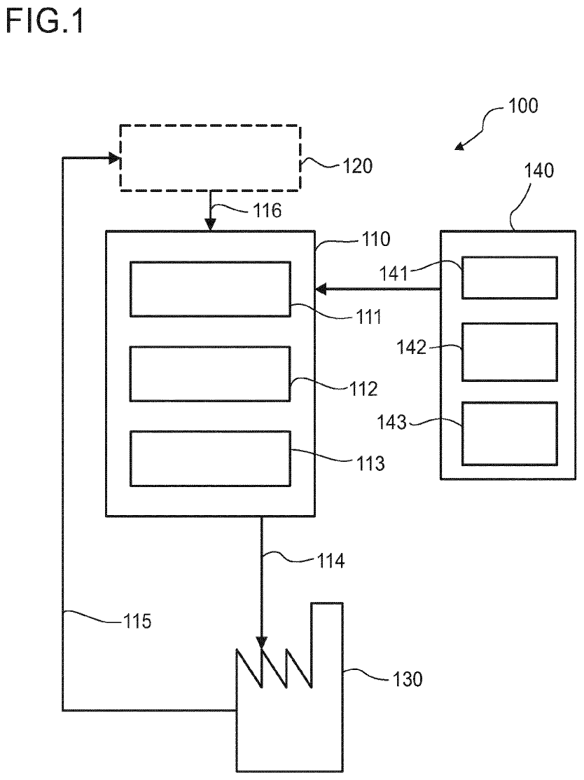

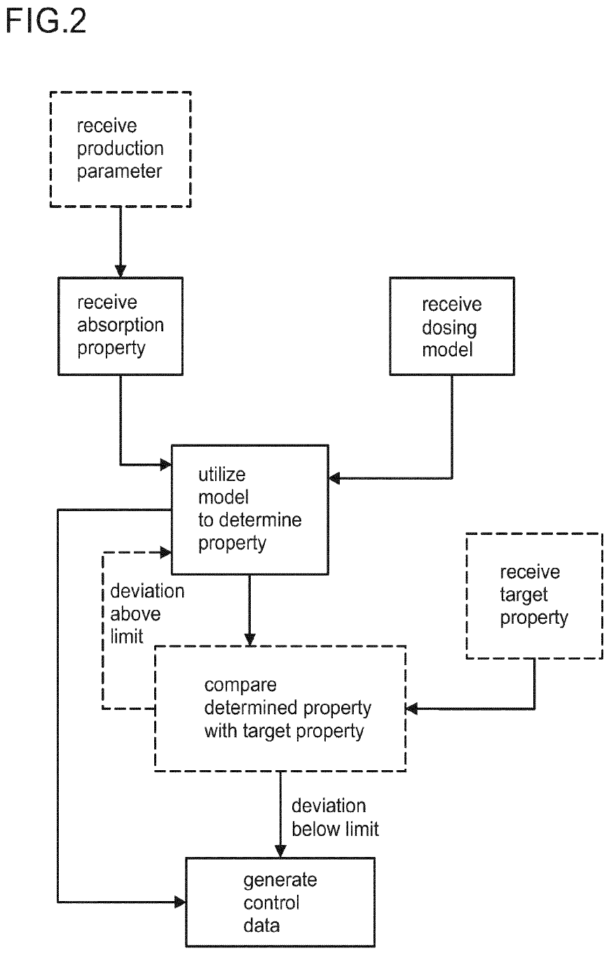

FIG. 1 shows schematically and exemplary a system 100 for controlling a production of a sanitary product and/or a superabsorbent material utilized in the sanitary product. The system 100 comprises an apparatus 110 for determining a hygiene property of a sanitary product. Further, the system comprises an interface unit 120 for interfacing with the production system 130 for producing the sanitary product and/or the superabsorbent material of the sanitary product. The apparatus 110 can be realized as any dedicated or general computing hardware comprising one or more processors. In particular, the apparatus 110 can be realized in a distributed computing, in which different functions of the apparatus are performed by different processors at the same or at different locations. The apparatus 110 is then realized by performing functions determined by the method described with respect to FIG. 2. Generally, the functions performed by the apparatus 110 can, for instance, be performed by a receiving unit 111, a hygiene property determination unit 112 and a control data generation unit 113.

The apparatus 110 is configured for determining a hygiene property for a sanitary product. The receiving unit 111 of the apparatus 110 is configured to receive one or more absorption properties of a superabsorbent material. For example, the receiving unit 111 can be configured to utilize a user interface into which a user can input a respective superabsorbent material that should be utilized in the sanitary product. Based on the input superabsorbent material, the receiving unit 111 can then be configured to access a storage unit or library on which absorption properties for a plurality of superabsorbent materials are already stored. Moreover, the receiving unit 111 can also directly receive the absorption properties of a superabsorbent material via an input interface from the user. Or a respective superabsorbent material with associated one or more absorption properties can be predetermined to be utilized without further notice for all cases. However, in a preferred embodiment, the system 100 can further comprise or be communicatively coupled to an apparatus 140 for determining an absorption property of a respective superabsorbent material.

The apparatus 140 comprises, for example, a receiving interface 141, one or more processors 142 and an output interface 143. Generally, the apparatus is configured to determine an absorption property of a superabsorbent material. The superabsorbent material is preferably utilized in form of superabsorbent particles comprising a superabsorbent polymer provided in form of an interconnected core and a surface cross-linked shell with a higher connectivity than the interconnected core. Generally, the apparatus 140 can be provided as a standalone device, for example, can be provided as a dedicated computing device, but can also be provided as part of a more general computing device providing additional functions. In particular, the apparatus can be provided as part of a quality control system or, for example, as part of the production control system.

The receiving interface 141 is configured in this example to receive a particle size of the superabsorbent particles of the superabsorbent material. Generally, the receiving interface can be realized as any interface that allows to receive respective data indicative of the particle size. In particular, the receiving interface can be configured to provide an interface to a storage unit on which a particle size is already stored. However, the apparatus 110 can also provide the particle size or a controlling system of the production system 130 can provide sensor measurements indicative of the particle size. The particle size can refer to any quantity that allows to quantify a volume of a particle of the superabsorbent particles of the superabsorbent material. Preferably, the particle size refers to a volume of the particle or if the particle can be approximated as a spherical particle, to a radius or diameter of the particle. The particle size of the superabsorbent particles generally is provided in a dry state of the superabsorbent particles, i.e., before the absorption of a fluid into the superabsorbent particles leading to an increase of the size of the superabsorbent particles. The received particle size of the superabsorbent particles is then provided to the one or more processors 142.

The one or more processors 142 are then configured to utilize an absorption property determination model for determining the absorption property of the superabsorbent material based on the particle size. For example, the one or more processors can be configured to access a storage unit on which the absorption property determination model is already stored. Generally, on a storage unit more than one absorption property determination model can be stored, for instance, property determination models for different superabsorbent materials produced in accordance with manufacturing specifications, for instance, using different superabsorbent polymers, different cross-linking methods and/or production parameters, can be stored. In this case, the one or more processors can then be configured to utilize respective information on the superabsorbent material, for instance, an ID of the superabsorbent material or a manufacturing specification provided for the superabsorbent material to select the respective absorption property determination model for the superabsorbent material. Moreover, for different absorption properties, different absorption property determination models can be stored on the storage unit. In this case, the one or more processors can be configured to either select all absorption property determination models available for a respective superabsorbent material and to then apply each of the absorption property determination models to determine all absorption properties available for the respective superabsorbent material, or based on further information on the desired absorption property, for example, provided via the user interface, the one or more processors can be configured to select the respective absorption property determination model to be utilized.

Generally, the absorption property determination model has been parameterized such that it is adapted to determine an absorption property of a superabsorbent particle based on the size of the particle. In particular, the data-driven determination model can be any machine learning based model that allows to learn based on historical data to determine an absorption property of a superabsorbent particle based on the size of the particle. For example, the absorption property determination model can refer to a regression model based algorithm like a neural network algorithm, a lasso algorithm, a ridge regression algorithm, a principal component based regression method, a robust multiple linear regression, a MASS algorithm or a random forest algorithm. However, the absorption property determination model can also refer to a classifier-based model algorithm like a random forest algorithm or an SVM algorithm. Useful algorithms are disclosed in “Introduction to Multivariate Statistical Analysis in Chemometrics, K. Varmuzza, P. Filzmoser, CRC Press, New York 2009” which is expressly incorporated in here by reference. In particular, it is preferred that the utilized absorption property determination model is further parameterized based on a core size and a shell size of a respective superabsorbent particles in order to allow to quantify and determine the respective contributions of the core size and the shell size to the respective absorption property. This allows to store the absorption property determination models by storing the respective performance parameters quantifying the contribution. A selection of the absorption property determination model can then be realized by selecting the performance parameters corresponding to a respective core size and respective shell size and using these performance parameters in the absorption property determination model.

Generally, the absorption property determination model can be trained in any known way. In particular, historical training data is utilized for training the absorption property determination model. The historical training data comprises at least two, preferably a plurality of particle sizes for a superabsorbent material and corresponding one or more measured absorption properties of the superabsorbent material. Such training data can be generated, for instance, by measurement of a respective superabsorbent material using known measurement methods for determining the absorption property and also for measuring the respective particle size of the superabsorbent material. Generally, such historical training data is often generated during a quality control of a superabsorbent material or during a design process of a superabsorbent material in which respective measurements are performed. The historical training data can then be utilized for parameterizing the absorption property determination model such that the parameterized absorption property determination model is adapted to determine the absorption property of the superabsorbent material based on the particle size. For example, known machine learning, i.e. parameterizing, methods can be utilized. Based on such a trained property determination model, the one or more processors 142 of apparatus 140 are then configured to determine an absorption property of the superabsorbent material based on the provided particle size. The determined absorption property and optionally also the utilized particle size can then be provided to apparatus 110. Thus, the apparatus for determining the absorption property of the superabsorbent materials allows to determine absorption properties also for superabsorbent materials that are not directly stored on a database or in a library. In particular, the influence of the particle size can be determined more accurately, in most cases, when utilizing the apparatus 140.

Further, the receiving unit 111 receives a dosing model for the sanitary product comprising the dosing of a predetermined liquid with time as a superabsorbent material layer of the sanitary product. Generally, the dosing model allows to specify the respective application for which the hygiene property of the sanitary product should be determined. Moreover, the dosing model can implicitly also take different compositions and structures of the sanitary product into account, in particular, compositions and structures that have an influence on fluid applied to the sanitary product reaching the superabsorbent material in the superabsorbent layer.

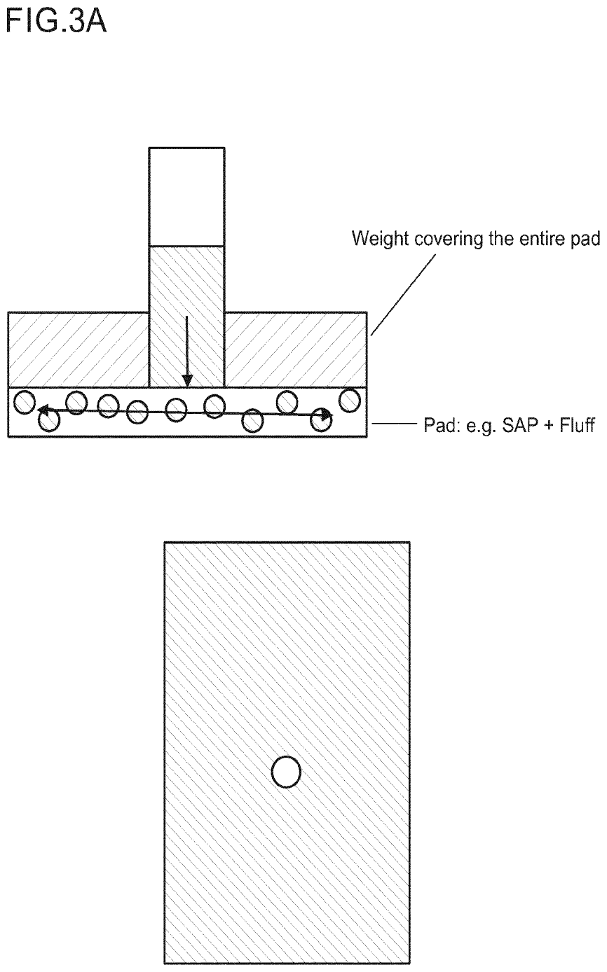

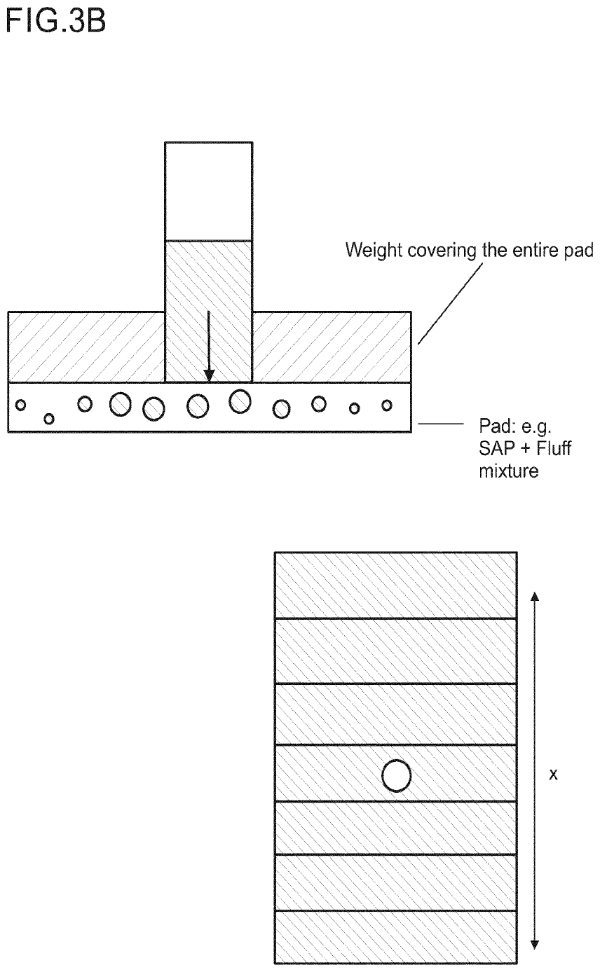

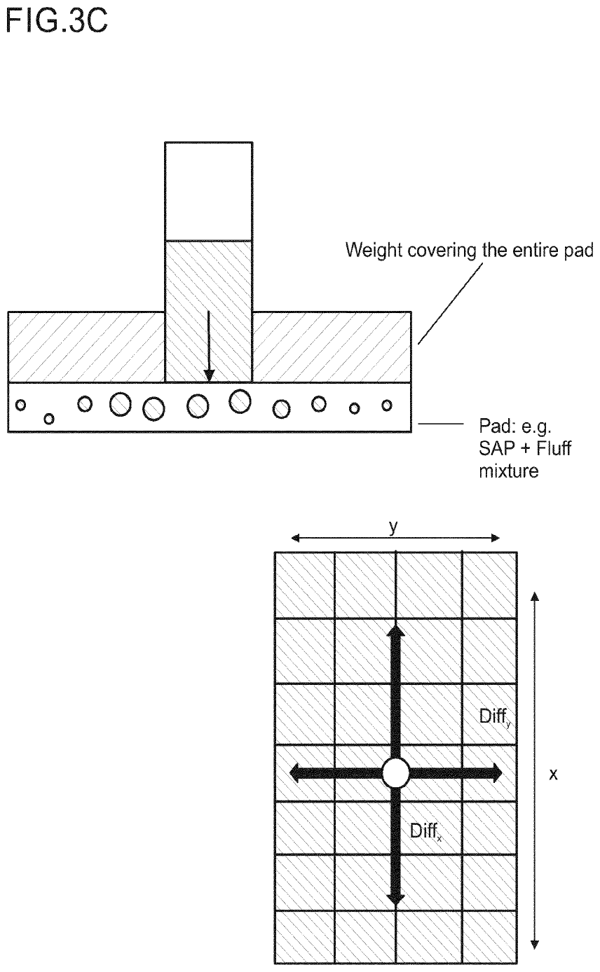

The hygiene property determination unit 112 is then configured to utilize a sanitary product property determination model for determining a hygiene property of the sanitary product based on the absorption properties and on the dosing model. A preferred example of such a product property determination model will be described in the following with respect to FIG. 3a.

FIG. 3a shows schematically and exemplary an illustration of a one-dimensional (1D) model for determining a hygiene property of a sanitary product. However, the model can also refer to more dimensions, for instance, can also be a 2D model or even a 3D model as shown in FIGS. 3b and 3c. Whereas in case of a 1D model ordinary differential equations comprising only functions of time can be used, in a 2D and 3D model differential partial equations which comprise functions of time t and spatial liquid distribution can be utilized. In a 2D-model the hygiene article can mathematically be segmented in stripes for describing a liquid distribution only in one direction, whereas in a 3D-model the liquid can be described as being distributed in two spatial orientations, e.g. a liquid distribution in x and γ-direction. Such 2D and 3D-models are exemplified in FIG. 3b and FIG. 3c. For the purpose of optimizing an absorption property of a superabsorbent polymer in a hygiene article it is in most cases sufficient to use a simple 1D-model, while for optimizing a hygiene article itself a 2D- or 3D-model is preferred.

The shown model in FIG. 3a is a 1D model showing, in this example, the layer comprising the superabsorbent material and optionally a fluff material. Further, the model illustrates the source of fluid for the dosing model, here a funnel reservoir. Moreover, in some cases the model further includes the effect of pressure provided to the superabsorbent material layer, as indicated by the shown weight. The arrow indicates the possible flow directions of fluid from the reservoir in the layer. In this model the free fluid in the layer can be described, for example, based on a differential equation comprising three terms, in particular a term indicating the dosing, a term indicating a liquid absorption, and a term indicating free liquid not yet absorbed. Preferably, the model is based on the differential equation

$$\frac{dL}{dt} = - \frac{dQ}{dt} + \frac{dV}{dt} ,$$

wherein

$$\frac{dL}{dt}$$

describes the free liquid in the sanitary product with time,

$$\frac{dQ}{dt}$$

describes the absorption of fluid by the superabsorbent material with time depending on the absorption properties, and

$$\frac{dV}{dt}$$

describes the dosing of the fluid with time into the sanitary product. The dosing term

$$\frac{dV}{dt}$$

can be expressed as the differential form of Torricelli's law as gravity dosing as used in many laboratory tests by which a target hygiene property in a sanitary product can be defined. In Torricelli's law an effective cross-section area A₂ bottlenecks the inflow of gush liquid into the sanitary product and represents the fluid permeability of this product under use, test, or simulation conditions. For example, the dosing term

$$\frac{dV}{dt}$$

can be expressed as:

$$\frac{dV}{dt} = {A}_{1} \times {v}_{1} = {A}_{2} \times {v}_{2} ,$$

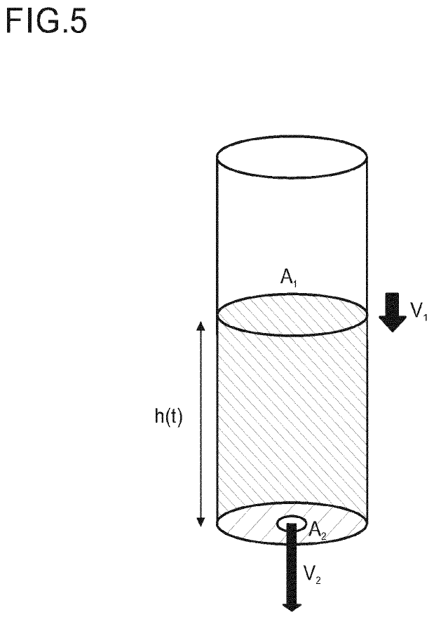

with A₂ the effective cross-section area for the pad inflow and A₁ the cylinder cross section area, v₁ and v₂ are the velocities of the liquid flow, as shown for all parameters in FIG. 5. Generally, A₂ can be set in accordance with the permeability properties of the sanitary product, in particular, taking the permeability of the superabsorbent material and surrounding structures into account. For example, for a high permeability area A₂ can be set higher than for a lower permeability of the sanitary product. v₂ depends on the gravity and the height of the liquid in the cylinder and can be calculated according to v₂=\sqrt{2gh} with h the height of the liquid in the cylinder and g the gravity constant. Based on this a time of liquid discharge also called acquisition time can be calculated according to

$${t}_{empty} = \frac{{A}_{1}}{{A}_{2}} \sqrt{\frac{2 {h}_{t = 0}}{g}} ,$$

With hₜ₌₀ the height of the liquid in the cylinder at the beginning of the dosing. Moreover, the acquisition time determines when the liquid is completely collected by the sanitary product and can thus also depend on the build of the hygiene product. Both the acquisition time and the free liquid can be optimized as hygiene properties of the hygiene product.

Alternatively, the dosing term may be any coded liquid dosing profile simulating a pump, or a natural gush, and may include the effects of liquid managing elements, or inefficiencies, observed in a sanitary product. In the present invention one or more subsequent liquid dosings may be simulated in a sanitary product to define one or more target hygiene properties. Examples for target hygiene properties are the amount of free liquid still present in a sanitary product at a certain time of the simulation and after a certain amount of liquid has been dosed. More than one time point may be defined for computing the free liquid amount and defining a target hygiene property. Another example of a target hygiene property is the acquisition time required to absorb all liquid in a sanitary product up to a preset threshold for one or more subsequent gushes. Another example of a target hygiene property is the maximum amount of free liquid available in a sanitary product after each gush. Yet another example of a target hygiene property is the integral of the available free liquid in a simulated sanitary product over time and for one or more subsequent gushes.

The absorption of the Fluid is preferably described by the following equation comprising absorption properties:

$$\frac{dQ}{dt} = \frac{1}{tau ( p )} * ( Qmax ( p ) - Q ( t ) )$$

Absorption properties in the above equation can be a free swell absorption capacity Q that can be determined, for instance, as CRC or FSC, or an absorption against pressure (AAP) if an external pressure is applied during the absorption process. Moreover, a characteristic absorption time tau can be utilized as absorption property to describe the time-dependency of the absorption process. Both, tau and Q can be functions of the external pressure p during the absorption process. Qmax refers to the maximum absorption capacity that equals the equilibrium swelling capacity after infinite or experimentally reasonable long swelling time. As permeability is typically inversely correlated to the equilibrium absorption capacity it can be indirectly derived for the purpose of optimization.

In the 1D model it is preferably assumed that the liquid is instantly equally distributed to all accessible superabsorbent material, and that all of the superabsorbent material in the layer has the same swelling degree. However, for this model it is not necessary that all of the superabsorbent material is accessible. In this model the absorption layer is not spatially discretized. FIG. 3b shows schematically and exemplary a model of the hygiene product in 2D. In this case the absorption layer is spatially discretized in one direction. Preferably, since most hygiene articles are provided with the absorption layer having a predetermined layer thickness and also being longer in one direction that in the other remaining direction, the discretization is performed in the longer direction of the layer. However, the discretization direction can also be performed in the direction in which the most changes in the superabsorbent layer are expected. In this 2D model it is preferably assumed that the liquid isotropically diffuses to all of the accessible superabsorbent material and that the superabsorbent material swells once liquid arrives at the superabsorbent material layer. FIG. 3c shows schematically and exemplary a model of the hygiene product in 3D. In this case the absorption layer is spatially discretized in two directions. Preferably, the two directions are the directions in which the superabsorbent layer extends on the surface of the hygiene product, i.e. the layer thickness is not discretized. In this case it is assumed that the liquid isotropically diffuses to all accessible superabsorbent material, and that the superabsorbent material swells once the liquid arrives at the superabsorbent material.

In the model only the superabsorbent properties, preferably, an absorption capacity under use case pressure, an absorption speed under use case pressure, or a permeability under use case pressure are considered. The dosing model, and thus the dosing of the fluid with time can, for example, be provided based on an experimental determination with a synthesized hygiene article, e.g. via a design of experiments in case of SAP-Fluff-mixtures. Preferably, the dosing model further takes the permeability of the superabsorbent material or a composite permeability of the mixture into account. This can be achieved, for instance, again through experimental measurements of the dosing but also by utilizing known physical relations between the permeability of a material and a possible fluid flow through the material, for instance, utilizing Toricelli's law. However, the dosing model can also be a theoretical dosing model that does not take the permeability or specific arrangements of the hygiene article into account. Such theoretical dosing models can be in particular useful for comparing different superabsorbent materials, in particular, the influence of different superabsorbent properties in a theoretical set up. The model can then calculate the time-dependent amount of not yet absorbed, i.e. free, liquid as a function of absorption capacity, amount of superabsorbent, time elapsed after acquisition is completed. The free liquid is a preferred hygiene property because it determines how much unabsorbed liquid is present at time t after a gush in the hygiene article and determines how dry the article is after a predetermined time after each liquid gush.

The experimental determination of the unabsorbed liquid (rewet) is tedious because it is experimentally done after a predetermined elapsed time t (t=1−20 min) after each gush via blotting paper placed on top of the hygiene article and applying a predetermined pressure to squeeze out this unabsorbed liquid. The amount of liquid dosed, the dosing sequence, and the time t for the rewet determination are variable and they all differ for different applications. As the experimental method can only deliver such rewet data at one fixed time per hygiene article and often only at the end of testing after the last gush, it requires for example two complete testing series to check two different t-values for one superabsorbent grade used at same concentration and in same hygiene article design. Hence, variation of superabsorbent grade, concentration of superabsorbent, dosing sequence of liquid, and rewet-test time (t) produce a significant amount of manual labor which slows down development and commercialization of optimized superabsorbents. The model, for example, as described above, enables to do such variations for example via a grid-search across a matrix of these properties quickly and with much less manual workload and time consumption. In contrast to the manual rewet-method it can produce time-dependent drying curves that enable to derive the optimum balance between absorption capacity, absorption speed, and liquid permeability for different rewet-time requirements t. To accomplish this the model can use experimental information on the absorption properties, e.g. absorption capacity, absorption speed, and liquid permeability, as functions of external use case pressure, determined on the pure superabsorbent by experiment, and stored in a data base.

Alternatively, at least absorption capacity and absorption speed under external use case pressure can be estimated via the theory described in F. L. Buchholz, A. T. Graham, Modern Superabsorbent Polymer Technology, Wiley-VCH, Weinheim, 1998. Moreover, as described above a respective data-driven model can also be used. In particular, particle size distribution effects on absorption capacity, absorption speed, and permeability can be modeled accurately using a data-driven model as already described above, as typically the absorption speed and permeability are non-linear functions of particle size mix and it is important in the production process to approximate a constant particle size distribution with as close as possible optimized performance balance of these parameters. In a preferred embodiment, the model is only based on the permeability and absorption speed.

The control data generation unit 113 can then generate control data for controlling a production process of the sanitary product and/or the superabsorbent material based on the determined hygiene property of the sanitary product. The control data are indicated in FIG. 1 by arrow 114. In a preferred embodiment, the control data are generated for direct controlling of the production process performed by production unit 130. In this case, via the interface unit 120, production parameters utilized for producing the sanitary product indicated by arrows 115 and 116 are provided to apparatus 110. In this case, the interface unit 120 is further configured to determine based on the one or more production parameters one or more absorption properties of the superabsorbent material and/or a dosing model for the sanitary product based on the received one or more production parameters. For example, the production parameters can indicate which superabsorbent material is utilized for the production of the sanitary product and the interface unit 120 can then utilize the same databases or libraries as the receiving unit 111. However, the interface unit 120 can also utilize the apparatus 140 for determining, for instance, based on a particle size of the superabsorbent material, the absorption properties utilizing the respective absorption property determination model. However, also other knowledge, for instance, other databases and libraries, associating one or more production properties, for instance, of a superabsorbent material, with respective absorption properties, or of a sanitary product with the respective dosing model can be utilized. However, the absorption properties of the superabsorbent material and/or the dosing model can also be provided, for instance, via an input unit from a user, to the interface unit. Further, the interface unit 120 can receive a target hygiene property for the sanitary product. The respectively received and/or determined parameters are then provided as indicated by arrow 116 to the apparatus 110, wherein the apparatus 110 can then predict based on the provided parameters, as described above, a hygiene property of the produced sanitary product. This allows to avoid measurements of the hygiene properties of the sanitary product during a running production at a plurality of times. Based on the determined hygiene property of the sanitary product, the apparatus 110, for instance, the control data generation unit 113, can then be configured for further comparing the determined hygiene property with the target hygiene property. In case that the determined hygiene property lies outside a predetermined range around the target hygiene property, control signals can then be generated such that the production parameters for producing the sanitary product and/or the superabsorbent material are directly amended. Generally, the amendment of the production parameters can be based on known rules, for instance, determined by experiment or experience that indicate that a deviation of a specific hygiene property can be corrected by incrementally amending respective production parameters.

Additionally or alternatively, the apparatus 110 can also utilize a target hygiene property for optimizing the sanitary product, in particular, before the production of the sanitary product, and then to provide respective control data for producing the optimized sanitary product. In this case, the apparatus 110, for instance, the control data generation unit 113, or the property determination unit 112, can further be configured for comparing a determined hygiene property with a target hygiene property. Based on the comparison, in particular, if the determined hygiene property meets the target hygiene property within predetermined limits, the respective utilized superabsorbent material, in particular, the utilized particle size distribution of the superabsorbent material, the composition of the superabsorbent material and the amount of the superabsorbent material, can be set as target superabsorbent material. However, in case the comparison results in that the determined hygiene property does not meet the target hygiene property within the predetermined limits, a new superabsorbent material can be determined and the determination of the hygiene property of the sanitary product can be repeated with the new superabsorbent material. This optimization can then be performed iteratively until either an abortion criterion is met, for instance, a predetermined number of iteration steps, or a superabsorbent material is found that can be set as a target superabsorbent material. The new superabsorbent material can be determined by amending, for instance, a size distribution of the particles forming the superabsorbent material, the composition of the superabsorbent material itself, and/or the amount of the superabsorbent material in the superabsorbent layer. Any of these variables for the sanitary product can have a respective influence on the absorption properties of the superabsorbent material and thus allows during the iteration to find a superabsorbent material that meets the target hygiene property.

FIG. 2 shows schematically and exemplarily a method for controlling a production process of a superabsorbent material and/or a sanitary product comprising the superabsorbent material. Generally, the method comprises steps of receiving absorption properties of the superabsorbent material and receiving a dosing model indicative of an application and/or a compositional structure of the sanitary product. Optionally, the receiving of the absorption properties is based on received production parameters of a production of a sanitary product, as descried, for instance, above with respect to FIG. 1. In a further step, the respective received absorption properties and received dosing model are then utilized for determining a hygiene property of the sanitary product. The utilized model can refer to a differential equation model as described above in more detail with respect to FIGS. 1 and 3. In an optional step, the method can comprise also receiving a target hygiene property and further to compare the determined hygiene property with the target hygiene property. Based on the comparison, it can then be determined to provide a new hygiene property, in particular, if a deviation above a predetermined limit is determined, and to again utilize the model for determining a hygiene property for the new superabsorbent material, or if the deviation lies below a predetermined limit, the respective superabsorbent material can be determined as a target superabsorbent material. Control data can then be generated either directly based on the determined hygiene property, for instance, for directly controlling a production process of a superabsorbent material or the sanitary product in order to meet respective target hygiene properties of the final product, or can be generated based on the target superabsorbent material, for instance, by indicating for the production process to utilize the respectively determined target superabsorbent material.

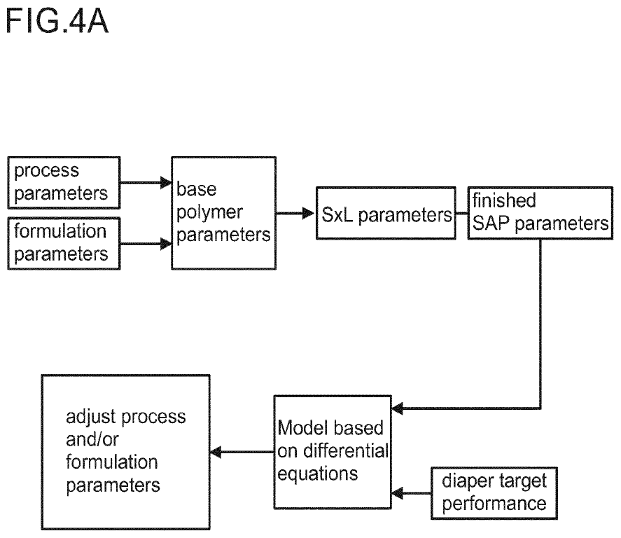

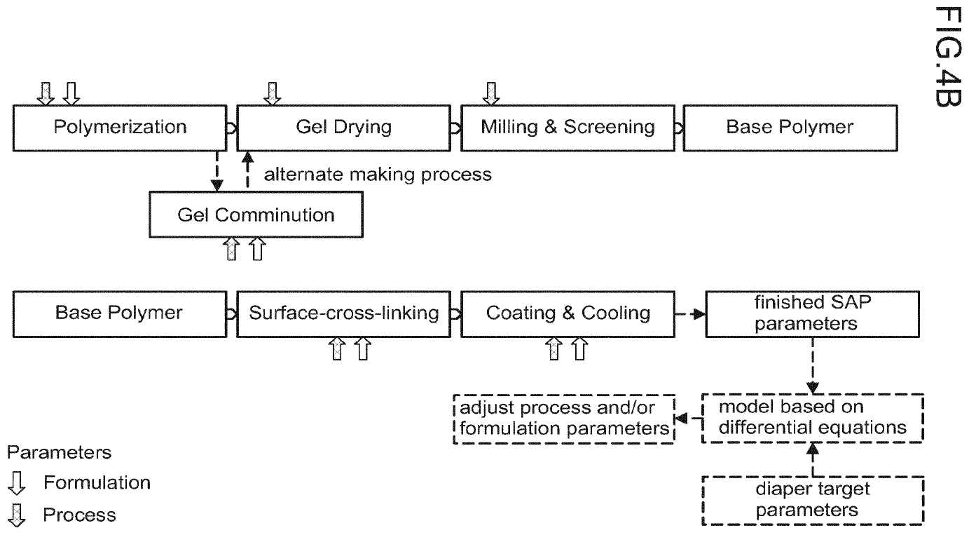

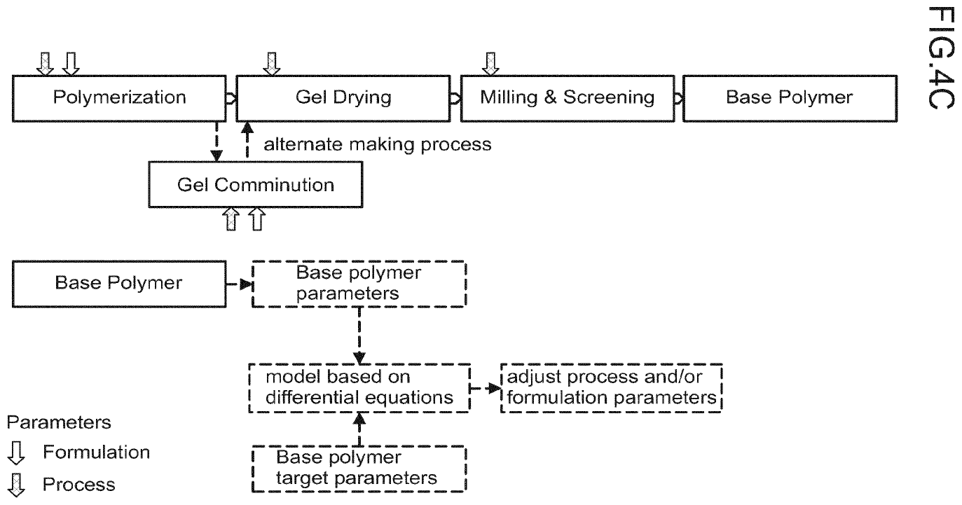

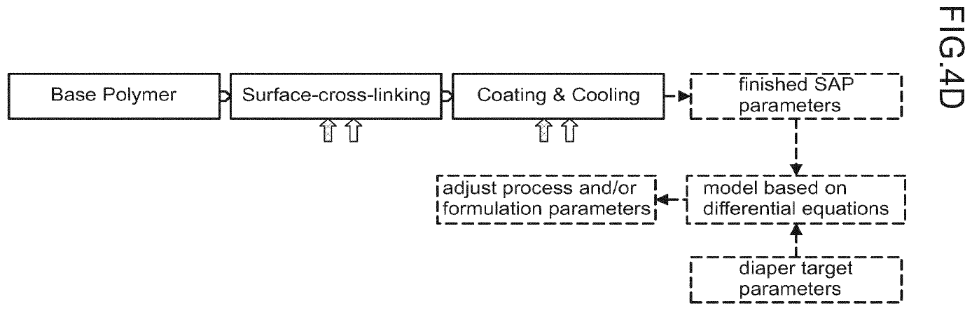

FIG. 4a shows schematically and exemplarily a more detailed example for the application of the above described invention to a specific sanitary product production. In particular, first process parameters like a polymerization, drying and sizing, and formulation parameters for a respective superabsorbent material are provided that allow to determine a respective base polymer that can be utilized for the production of superabsorbent particles. The base polymer utilized for the production of superabsorbent particles comprises as characteristics a capacity, swelling time, particle size distribution and particle morphology. Based on these characteristics, further a formulation and production process with respective parameters can be determined referred to as SXL parameters. This complete production process then leads to a finished superabsorbent particle production, wherein the superabsorbent particles comprise respective absorption properties, in particular, a capacity, a swelling time and a permeability. These superabsorbent particle parameters together with diaper target performance parameters indicative of target hygiene properties of a diaper, like free liquid after a predetermined amount of time, can then be provided to a hygiene property determination model based on differential equations as described above, for instance, with respect to FIG. 3. The model then allows to compute the diaper performance, in particular, the hygiene properties, from the superabsorbent particle parameters and to compare the computed diaper performance with the target performance. Based on this comparison, the process and formulation parameters can then be adjusted, utilizing, for instance, machine learning, white-box models, or a combination thereof for optimizing the sanitary product, in this case the diaper, to meet the target performance.

FIGS. 4b to 4d show in more detail the superabsorbent particle production and at which steps of the production the parameters can be optimized based on the target performance. In FIG. 4b the optimization process is configured to optimize the superabsorbent material production over all production steps of the production of the superabsorbent material. The arrow at respective steps indicate if in this step the process parameters or the formulation parameters can be amended for the optimization of the superabsorbent material. Generally, the optimization can be performed as described with respect to FIG. 4a utilizing hygiene property determination model based on differential equations as described above, for instance, with respect to FIG. 3, to determine respective hygiene properties and to compare the hygiene properties with the respective target performance, i.e. with target hygiene properties. In FIG. 4c an optimization process is shown in which only the steps of producing the superabsorbent material without the surface adaptation, e.g. without the surface cross-linking and/or coating steps, are optimized. Moreover, in this example, the hygiene property determination model has already determined the optimal target absorption properties of the superabsorbent material that allow to provide the respective target performance of the hygiene product. Thus, in this case the optimization can be performed directly by comparing the absorption properties of the superabsorbent material determined, for instance, utilizing a respective absorption property determination model as also described above, with the target absorption properties. In FIG. 4d a further optimization process is shown in which only the last steps of the superabsorbent material production referring to the surface adaption, for instance, to the cross-linking, is shown. Also, in this case it is exemplarily shown that the optimization can be directly performed based on target absorption properties of the absorption material determined previously with respect to a target performance of a hygiene product.

Although in the above embodiments the invention has been described utilizing a model based on differential equations, also other models can be utilized, in particular, data driven models realized as machine learning models that are trained on respective historical training data.

For improving diaper performance and developing/supplying adequate superabsorbents it is required to understand how the many different performance parameters affect good diaper performance. This has been historically done by trial and error as well as incremental development activities. Due to increasing market complexity, diaper design evolution, and increased commoditization it is now required to develop methods to drive innovation more effective and efficient. Therefore, computational methods are required that enable definition of performance critical SAP-parameters and selection of optimized parameter sets in superabsorbents to enable fast development of superabsorbents that will result in improved diaper performance.

The above invention allows, inter alia, improve the balance between absorption capacity, absorption speed, and acquisition speed that needs to be optimized to fit certain application tests and/or consumer preferences. The present invention enables more effective decisions in the development of new hygiene products.

In the description above a plurality of absorption properties are described that are in most cases defined by a respective measurement or test method utilized for determining the absorption property. Respective sources for details in these measurements and test methods are provided in the following. Generally, various methods to determine absorption properties for superabsorbents are available as industry standards, in publications, are specified by a user application, or are widely used in the markets by experience. For example, method for determining the FSC, CRC, AAP properties are available as EDANA methods from EDANA the European disposables and non-wovens association. In particular, for the FSC property the measurement method is described in the standard NWSP 240.0.R2 (15), for the CRC property the measurement method is described in the standard NWSP 241.0.R2 (15) and for the AAP property the measurement method is described in the standard NWSP 242.0.R2 (15). Further, China country test methods are available from Standardization administration of the PRC. Further, a Saline Flow Conductivity (SFC) measurement method is disclosed, for example, in the patent disclosure EP 0752892 B1. A volumetric absorbency under load (VAUL) measurement method is disclosed, for instance, in the patent disclosure EP 2922882 B1. A T20 absorption kinetics measurement method is disclosed, for example, in the patent disclosure EP 2535698A₁. Moreover, a vortex absorption kinetics measurement method is disclosed exemplary in the article “Modern Superabsorbent Polymer Technology.” By Buchholz FL and Graham AT, 1st ed. Weinheim: Wiley-VCH, 1998, p. 155. Further, an automated AAP measurement method is disclosed in the patent application WO 2021/001221 A₁.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Procedures like the receiving of the absorption properties, the determining of the hygiene property, the generating of the control data, etc. performed by one or several units or devices can be performed by any other number of units or devices. These procedures can be implemented as program code means of a computer program and/or as dedicated hardware.

A computer program product may be stored/distributed on a suitable medium, such as an optical storage medium or a solid-state medium, supplied together with or as part of other hardware, but may also be distributed in other forms, such as via the Internet or other wired or wireless telecommunication systems.

Any units described herein may be processing units that are part of a classical computing system. Processing units may include a general-purpose processor and may also include a field programmable gate array (FPGA), an application specific integrated circuit (ASIC), or any other specialized circuit. Any memory may be a physical system memory, which may be volatile, non-volatile, or some combination of the two. The term “memory” may include any computer-readable storage media such as a non-volatile mass storage. If the computing system is distributed, the processing and/or memory capability may be distributed as well. The computing system may include multiple structures as “executable components”. The term “executable component” is a structure well understood in the field of computing as being a structure that can be software, hardware, or a combination thereof. For instance, when implemented in software, one of ordinary skill in the art would understand that the structure of an executable component may include software objects, routines, methods, and so forth, that may be executed on the computing system. This may include both an executable component in the heap of a computing system, or on computer-readable storage media. The structure of the executable component may exist on a computer-readable medium such that, when interpreted by one or more processors of a computing system, e.g., by a processor thread, the computing system is caused to perform a function. Such structure may be computer readable directly by the processors, for instance, as is the case if the executable component were binary, or it may be structured to be interpretable and/or compiled, for instance, whether in a single stage or in multiple stages, so as to generate such binary that is directly interpretable by the processors. In other instances, structures may be hard coded or hard wired logic gates, that are implemented exclusively or near-exclusively in hardware, such as within a field programmable gate array (FPGA), an application specific integrated circuit (ASIC), or any other specialized circuit. Accordingly, the term “executable component” is a term for a structure that is well understood by those of ordinary skill in the art of computing, whether implemented in software, hardware, or a combination. Any embodiments herein are described with reference to acts that are performed by one or more processing units of the computing system. If such acts are implemented in software, one or more processors direct the operation of the computing system in response to having executed computer-executable instructions that constitute an executable component. Computing system may also contain communication channels that allow the computing system to communicate with other computing systems over, for example, network. A “network” is defined as one or more data links that enable the transport of electronic data between computing systems and/or modules and/or other electronic devices. When information is transferred or provided over a network or another communications connection, for example, either hardwired, wireless, or a combination of hardwired or wireless, to a computing system, the computing system properly views the connection as a transmission medium. Transmission media can include network and/or data links which can be used to carry desired program code means in the form of computer-executable instructions or data structures and which can be accessed by a general-purpose or special-purpose computing system or combinations. While not all computing systems require a user interface, in some embodiments, the computing system includes a user interface system for use in interfacing with a user. User interfaces act as input or output mechanism to users for instance via displays.

Those skilled in the art will appreciate that at least parts of the invention may be practiced in network computing environments with many types of computing system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, datacenters, wearables, such as glasses, and the like. The invention may also be practiced in distributed system environments where local and remote computing system, which are linked, for example, either by hardwired data links, wireless data links, or by a combination of hardwired and wireless data links, through a network, both perform tasks. In a distributed system environment, program modules may be located in both local and remote memory storage devices.

Those skilled in the art will also appreciate that at least parts of the invention may be practiced in a cloud computing environment. Cloud computing environments may be distributed, although this is not required. When distributed, cloud computing environments may be distributed internationally within an organization and/or have components possessed across multiple organizations. In this description and the following claims, “cloud computing” is defined as a model for enabling on-demand network access to a shared pool of configurable computing resources, e.g., networks, servers, storage, applications, and services. The definition of “cloud computing” is not limited to any of the other numerous advantages that can be obtained from such a model when deployed. The computing systems of the figures include various components or functional blocks that may implement the various embodiments disclosed herein as explained. The various components or functional blocks may be implemented on a local computing system or may be implemented on a distributed computing system that includes elements resident in the cloud or that implement aspects of cloud computing. The various components or functional blocks may be implemented as software, hardware, or a combination of software and hardware. The computing systems shown in the figures may include more or less than the components illustrated in the figures and some of the components may be combined as circumstances warrant.

Any reference signs in the claims should not be construed as limiting the scope.

1. An apparatus for determining a hygiene property for a sanitary product, wherein the sanitary product comprises a layer of superabsorbent material provided in a form of superabsorbent particles, wherein the apparatus comprises one or more processors configured to
receive a) one or more absorption properties of a superabsorbent material, and b) a dosing model for the sanitary product comprising a dosing of a predetermined liquid with time at the superabsorbent material layer of the sanitary product,
utilize a sanitary product property determination model for determining a hygiene property of the sanitary product based on the absorption properties and on the dosing model, and
generate control data for controlling a production process of the sanitary product and/or the superabsorbent material based on the determined hygiene property of the sanitary product.

2. The apparatus according to claim 1, wherein the one or more processors are further configured to receive a target hygiene property of the sanitary product, to compare the determined hygiene property with the target hygiene property, and to determine i) the superabsorbent material as a target superabsorbent material for the sanitary product, if the determined hygiene property lies within a predetermined range around the target hygiene property, and ii) a new superabsorbent material and repeating the determination of the hygiene property of the sanitary product using the new superabsorbent material if the determined hygiene property lies outside a predetermined range around the target hygiene property, and wherein the controlling of production process is based on the determined target superabsorbent material.

3. The apparatus according to claim 2, wherein determining of a new superabsorbent material comprises amending a size distribution of the particles forming the superabsorbent material and/or an amount of superabsorbent material in the layer.

4. The apparatus according to claim 1, wherein the hygiene property is indicative of an amount of free liquid in the sanitary product.

5. The apparatus according to claim 1, wherein the one or more absorption properties of the superabsorbent material are indicative of at least one of a permeability, an absorption capacity, and absorption speed.

6. The apparatus according to claim 4, wherein the sanitary product property determination model comprises solving the following differential equation
$$\frac{dL}{dt} = - \frac{dQ}{dt} + \frac{dV}{dt} ,$$
wherein
$$\frac{dL}{dt}$$
describes the free liquid in the sanitary product with time,
$$\frac{dQ}{dt}$$
describes an absorption of fluid by the superabsorbent material with time depending on the absorption properties, and
$$\frac{dV}{dt}$$
describes a dosing of the fluid with time into the sanitary product.

7. The apparatus according to claim 1, wherein further an indication on an amount of superabsorbent material in the sanitary product layer is received and wherein the one or more absorption properties of the superabsorbent material are adapted based on the amount of superabsorbent material in the sanitary product.

8. The apparatus according to claim 1, wherein the apparatus further comprises one or more processors that are configured for determining the one or more absorption properties of the superabsorbent material based on a received particle size distribution of the superabsorbent material utilizing an absorption property determination model, wherein the absorption property determination model is a data-driven model that has been parameterized such that the data-driven model is adapted to determine a technical application property of a superabsorbent particle based on the size of the superabsorbent particle, wherein the determined one or more absorption properties are then utilized in the sanitary product property determination model.

9. The apparatus according to claim 8, wherein the superabsorbent particles of the superabsorbent material comprise a superabsorbent polymer provided in a form of i) an interconnected core and ii) a surface crosslinked shell with a higher connectivity than the interconnected core, and wherein the provided absorption property determination model is further parameterized based on a core size and a shell size of the superabsorbent particles.

10. The apparatus according to claim 1, wherein the one or more received absorption properties are measured during a production process of the superabsorbent material and/or of the sanitary product comprising the superabsorbent material and wherein the generated control data is configured to control and/or monitor the production process based on the hygiene property determined from the one or more measured absorption properties.

11. A system for controlling a production of a sanitary product and/or a superabsorbent material, wherein the sanitary product comprises a layer of the superabsorbent material provided in form of superabsorbent particles, wherein the system comprises:
an interface unit configured for receiving a) one or more production parameters utilized for producing the sanitary product and b) a target hygiene property of the sanitary product, and for determining one or more absorption properties of the superabsorbent material and/or a dosing model for the sanitary product based on the received one or more production parameters,
an apparatus according to claim 1, wherein the apparatus is configured to receive the absorption properties of the superabsorbent material and/or a dosing model from the interface unit and to determine the hygiene property, and further to compare the determined hygiene property with the target property and if the determined hygiene property lies outside a predetermined range around the target hygiene property generate control signals for amending one or more of the one or more production parameters utilized for producing the sanitary product and/or the superabsorbent material.

12. A computer implemented method for determining a hygiene property for a sanitary product, wherein the sanitary product comprises a layer of superabsorbent material provided in form of superabsorbent particles, wherein the method comprises the steps of:
receiving a) one or more absorption properties of a superabsorbent material, and b) a dosing model for the sanitary product comprising the dosing of a predetermined liquid with time at the superabsorbent material layer of the sanitary product,
utilizing a sanitary product property determination model for determining a hygiene property of the sanitary product based on the absorption properties and on the dosing model, and
generating control data for controlling the production process of the sanitary product and/or the superabsorbent material based on the determined hygiene property of the sanitary product.

13. A computer implemented method for controlling a production of a sanitary product and/or a superabsorbent material, wherein the sanitary product comprises a layer of the superabsorbent material provided in a form of superabsorbent particles, wherein the method comprises:
receiving a) one or more production parameters utilized for producing the sanitary product and b) a target hygiene property of the sanitary product,
determining one or more absorption properties of the superabsorbent material and/or a dosing model for the sanitary product based on the received one or more production parameters,
performing the method according to claim 12 based on the received absorption properties of the superabsorbent material and/or a dosing model to determine the hygiene property, and,
comparing the determined hygiene property with the target property and if the determined hygiene property lies outside a predetermined range around the target hygiene property generate control signals for amending one or more of the one or more production parameters utilized for producing the sanitary product and/or the superabsorbent material.

14. A computer program product for determining a hygiene property for a sanitary product, wherein the computer program product comprises program code means for causing the apparatus of claim 1 to execute a method comprising the steps of:
receiving a) one or more absorption properties of a superabsorbent material, and b) a dosing model for the sanitary product comprising the dosing of a predetermined liquid with time at the superabsorbent material layer of the sanitary product,
utilizing a sanitary product property determination model for determining a hygiene property of the sanitary product based on the absorption properties and on the dosing model, and
generating control data for controlling the production process of the sanitary product and/or the superabsorbent material based on the determined hygiene property of the sanitary product.

15. A computer program product for controlling a production of a sanitary product and/or a superabsorbent material, wherein the computer program product comprises program code means for causing the apparatus of claim 11 to execute a method comprising the steps of:
receiving a) one or more production parameters utilized for producing the sanitary product and b) a target hygiene property of the sanitary product,
determining one or more absorption properties of the superabsorbent material and/or a dosing model for the sanitary product based on the received one or more production parameters,
performing a method based on the received absorption properties of the superabsorbent material and/or a dosing model to determine the hygiene property, comprising the steps of:
receiving a) one or more absorption properties of a superabsorbent material, and b) a dosing model for the sanitary product comprising the dosing of a predetermined liquid with time at the superabsorbent material layer of the sanitary product,
utilizing a sanitary product property determination model for determining a hygiene property of the sanitary product based on the absorption properties and on the dosing model, and
generating control data for controlling the production process of the sanitary product and/or the superabsorbent material based on the determined hygiene property of the sanitary product, and
comparing the determined hygiene property with the target property and if the determined hygiene property lies outside a predetermined range around the target hygiene property generate control signals for amending one or more of the one or more production parameters utilized for producing the sanitary product and/or the superabsorbent material.
